(19) United States
(12) Patent Application Publication
(10) Pub. No.: US 2024/0005361 A1
(43) Pub. Date: Jan. 4, 2024

(54) ELECTRONIC MEDICAL RECORD ADVERTISING PLATFORM METHOD AND DEVICES

(71) Applicant: DOCEREE INC, Parsippany, NJ (US)

(72) Inventor: Harshit Jain, Noida (IN)

(21) Appl. No.: 17/857,013

(22) Filed: Jul. 3, 2022

(51) Int. Cl. G06Q 30/02; G16H 10/60; G16H 40/20

(52) U.S. Cl. CPC G06Q 30/0275; G06Q 30/0271; G16H 10/60; G16H 40/20

(57) ABSTRACT

The embodiments disclose an apparatus including a network platform configured for receiving advertiser real-time bids for electronic health record system workflow webpage ad placement, at least one webpage graphic processor coupled to the network platform configured for integrating ad placement into electronic health record system workflow webpage formats, a data mining processor for extracting patient privacy regulatory compliant clinical triggers from a patient electronic health record, at least one electronic publishing device coupled to the network platform configured for displaying workflow webpage ads and messaging within each workflow webpage, and an electronic medical record advertising application wirelessly coupled to the network platform computer configured for accessing an electronic health record system having integrated targeted ads for electronic medical record advertising.

### BACKGROUND

Physicians can deliver better care with relevant information but lack timely access. Healthcare is experiencing significant digital transformation. But even with all the digitalization, health care providers do not have access to up-to-date, high-quality medical information that is only available from life sciences organizations. Research has proven that a health care provider's access to quality medical information can lead to optimal treatment outcomes.

### BRIEF DESCRIPTION OF THE DRAWINGS

FIG. 1 shows for illustrative purposes only an example of an overview of an electronic medical record advertising platform of one embodiment.

FIG. 2 shows a block diagram of an overview flow chart of targeting life science advertiser/supplier products of one embodiment.

FIG. 3 shows a block diagram of an overview of a winning bid selection processor of one embodiment.

FIG. 4 shows a block diagram of an overview of an electronic medical record advertising platform of one embodiment.

FIG. 5 shows for illustrative purposes only an example of a patient calendar of one embodiment.

FIG. 6 shows for illustrative purposes only an example of a patient chart of one embodiment.

FIG. 7 shows a block diagram of an overview of providing a physician with information of one embodiment.

FIG. 8 shows a block diagram of an overview of creation of ad copy in various formats of one embodiment.

FIG. 9 shows a block diagram of an overview of providing latest medical information to health care providers of one embodiment.

FIG. 10 shows a block diagram of an overview of patient affordability messaging of one embodiment.

FIG. 11 shows a block diagram of an overview of clinical alerts of one embodiment.

FIG. 12 shows a block diagram of an overview of disease awareness messaging of one embodiment.

FIG. 13 shows a block diagram of an overview of ads and information integrated within the practice workflow of one embodiment.

FIG. 14 shows a block diagram of an overview of selecting precise message based on the workflow of one embodiment.

FIG. 15 shows a block diagram of an overview of outcomes of one embodiment.

FIG. 16 shows a block diagram of an overview of HIPAA compliance of one embodiment.

FIG. 17 shows a block diagram of an overview of engaging with health care providers of one embodiment.

FIG. 18 shows a block diagram of an overview of an onboarding process of one embodiment.

FIG. 19 shows a block diagram of an overview of partnering of one embodiment.

FIG. 20 shows a block diagram of an overview of targeted EHR webpages to bid on of one embodiment.

FIG. 21 shows a block diagram of an overview of establishing health care provider workflow web pages of one embodiment.

FIG. 22 shows a block diagram of an overview of physician e-prescribing medication from product advertiser of one embodiment.

### DETAILED DESCRIPTION OF THE INVENTION

In the following description, reference is made to the accompanying drawings, which form a part hereof, and in which is shown by way of illustration a specific example in which the invention may be practiced. It is to be understood that other embodiments may be utilized, and structural changes may be made without departing from the scope of the present invention.

### General Overview

It should be noted that the descriptions that follow, for example, in terms of electronic medical record advertising platform method and devices are described for illustrative purposes and the underlying system can apply to any number and multiple types of clinical triggers. In one embodiment of the present invention, the electronic medical record advertising platform method and devices can be configured using electronic health records. The electronic medical record advertising platform method and devices can be configured to include clinical alerts integrated into the electronic health records and can be configured to include disease awareness messaging integrated into the electronic health records using the present invention. Wherein clinical triggers are defined as a specific event or condition that causes a particular outcome.

The terms “health care provider”, “provider”, “physician”, “doctor”, “Dr.”, “nurses”, other health care worker titles and “user” and “users” are used herein interchangeably without any change in meaning.

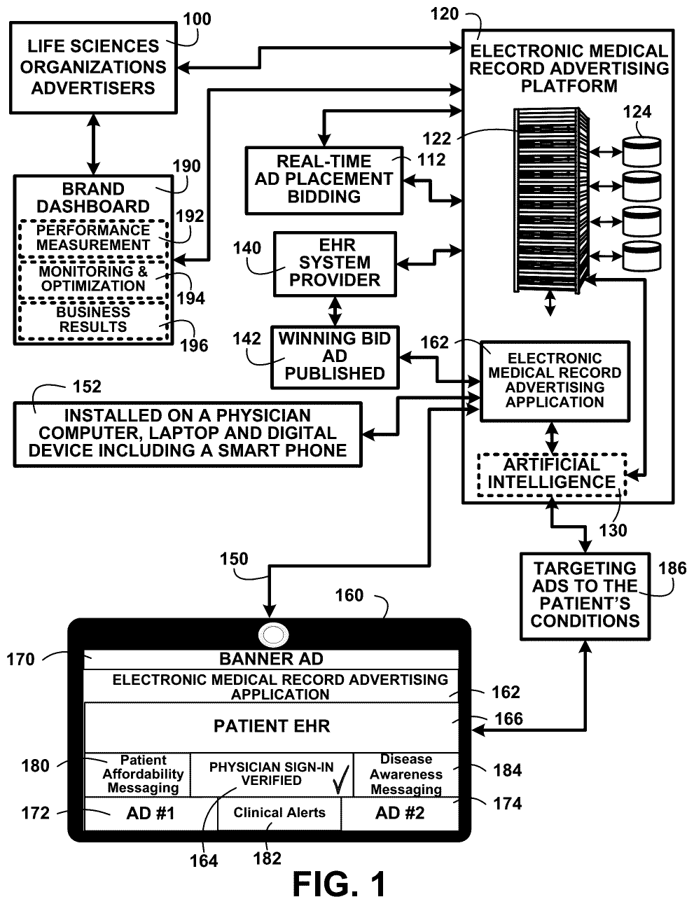

FIG. 1 shows for illustrative purposes only an example of an overview of an electronic medical record advertising platform of one embodiment. FIG. 1 shows in one embodiment, for example a life sciences organizations advertisers 100 participating in real-time ad placement bidding 112 through an electronic medical record advertising platform 120. The electronic medical record advertising platform 120 comprises a plurality of servers 122, a plurality of databases 124, a computer having an electronic medical record advertising application 162, and artificial intelligence 130 for analysis of data. In one embodiment, for example the selection of a winning bid ad published 142 for the EHR system is performed by an electronic health record (EHR) system provider 140 and the electronic medical record advertising platform 120.

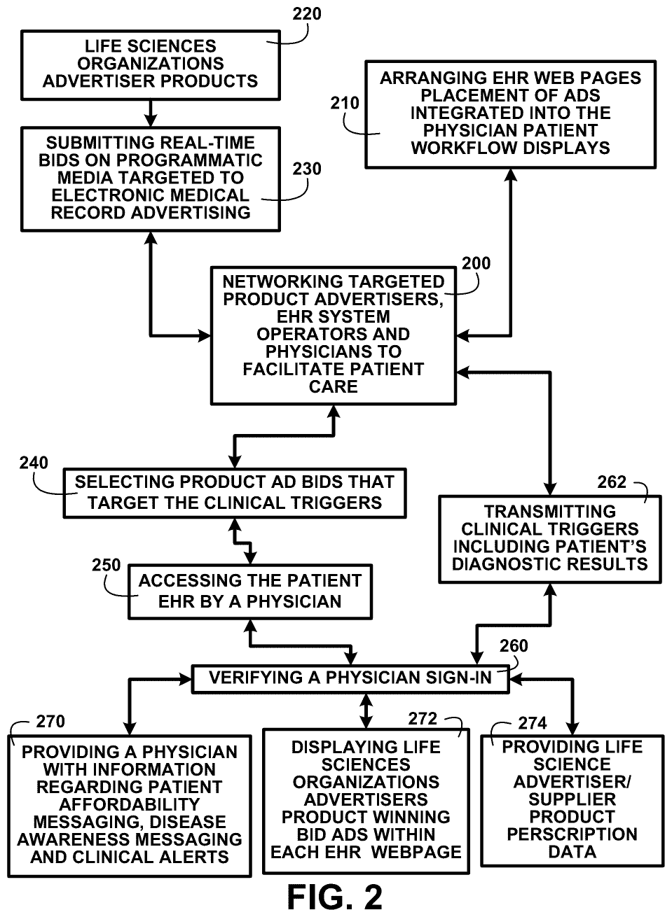

In one embodiment, for example the winning bid ad published 142 is transmitted to the physician 150 with the electronic medical record advertising platform 120. In one embodiment, for example the winning bid ad published 142 is transmitted with the electronic medical record advertising application 162 to a physician's digital tablet 160, during the physician's workflow with a patient. In one embodiment, for example the physician's verified log-in to the electronic medical record advertising platform 120 with the electronic medical record advertising application 162 on the physician tablet 160 displays a patient EHR 166. In other embodiments the electronic medical record advertising application 162 can be installed on a physician computer, laptop and digital device including a smart phone 152 allowing the physician to review the patient EHR, clinical triggers, lab results, disease awareness messaging, patient affordability messaging 180, clinical alerts 182, prescriptions of life sciences organizations advertisers 100 products, life science advertiser/supplier product prescription data 274 of FIG. 2 and the e-prescribing of advertiser products.

The EHR system webpages shows a banner ad 170 on the top edge and below ads at both corners including ad #1 172 and ad #2 174. The physician can advise the patient on coupons and other cost saving with patient affordability messaging 180 displayed on the physician tablet 160 when tapped by the physician. Tapping the clinical alerts 182 tab provides the physician with up-to-date concerns expressed by life sciences organizations for the physician's diagnosis.

Both the physician and the patient have available disease awareness messaging 184 to be better informed about any new and improved treatments and symptoms of disease mutations and potential side effects of available medications. The life sciences organizations advertisers 100 is targeting ads to the patient's conditions 186 to be a relevant source of information and present the latest medications and treatments available for the specific patient's conditions 186.

The electronic medical record advertising platform 120 tracks and records the EHR system webpages ad hits, messaging traffic and prescriptions of life sciences organizations advertisers 100 products. The tracking data is made available to the life sciences organizations advertisers 100 on an electronic medical record advertising application 162 brand dashboard 190 to keep the life sciences organizations advertisers 100 up to date on a performance measurement 192 for the ads placed, monitoring & optimization 194 of the effectiveness of the ads and the business results 196 of the targeted advertising on the physician EHR system of one embodiment.

### Detailed Description

FIG. 2 shows a block diagram of an overview flow chart of targeting life science advertiser/supplier products of one embodiment. FIG. 2 shows a collaboration of networking targeted product advertisers, EHR system operators, and physicians to facilitate patient care 200 facilitated by the electronic medical record advertising platform 120 of FIG. 1. The electronic medical record advertising platform 120 of FIG. 1 is arranging EHR web pages placement of ads integrated into the physician patient workflow displays 210. The suppliers' life sciences organizations advertiser products 220 targeted to health care providers patient conditions.

The product suppliers are submitting real-time bids on programmatic media targeted to electronic medical record advertising 230. The EHR system providers and electronic medical record advertising platform 120 of FIG. 1 join in selecting product ad bids that target the clinical triggers 240. The targeted ads are displayed when accessing the patient EHR by a physician 250. EHR system is verifying a physician sign-in 260 and through the electronic medical record advertising platform 120 of FIG. 1 extracting using a data mining processor and transmitting clinical triggers including patient's diagnostic results 262 and safeguarding privacy in compliance with the Health Insurance Portability and Accountability Act of 1996 (HIPAA).

The displayed information includes integrated targeted ads that provide information of new and effective treatments and additionally are providing a physician with information regarding patient affordability messaging, disease awareness messaging and clinical alerts 270. Displaying using at least one electronic publishing device the life science advertiser/supplier product winning bid ads within each EHR webpage 272 are also providing accessible life science advertiser/supplier product prescription data 274 the physician can utilize in prescribing medications for the patient of one embodiment.

### A Winning Bid Selection Processor

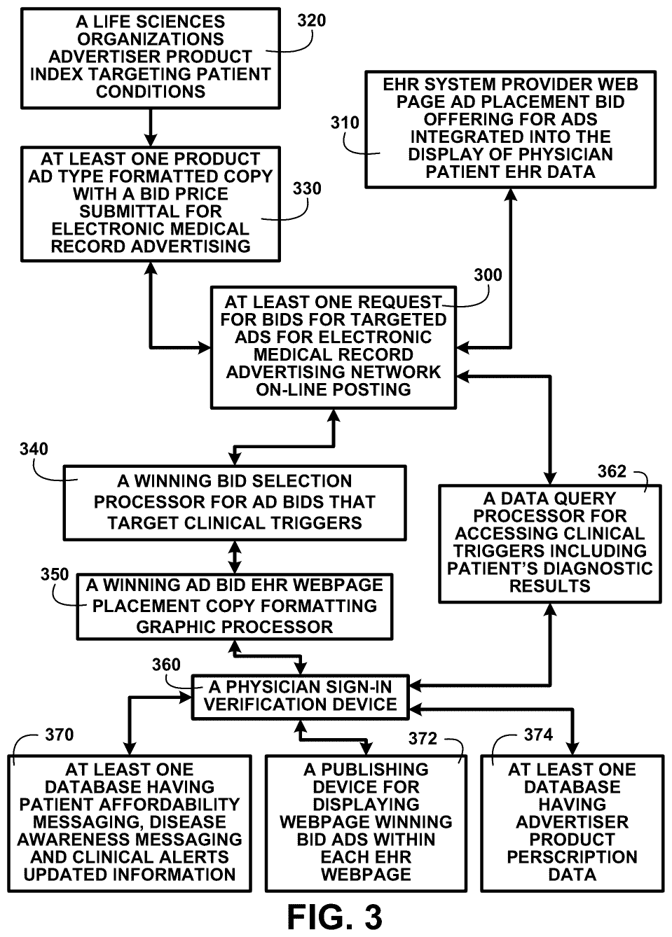

FIG. 3 shows a block diagram of an overview of a winning bid selection processor of one embodiment. FIG. 3 shows at least one request for bids for targeted ads for electronic medical record advertising network on-line posting 300. The bids are for EHR system provider web page ad placement bid offering for ads integrated into the display of physician patient EHR data 310. Bidding will be made by a life sciences organizations advertiser product index targeting patient conditions 320.

The life sciences organizations advertiser will submit at least one product ad type formatted copy with a bid price submittal for electronic medical record advertising 330. A winning bid selection processor for ad bids that target clinical triggers 340 will analyze also use a winning ad bid EHR webpage placement copy formatting graphic processor 350 for conformity in ad placement size and any reformatting needed to fit the web page layout. A physician sign-in verification device 360 will confirm the physician as licensed and a subscriber to the EHR system.

A data query processor for accessing clinical triggers including patient's diagnostic results 362 queries the EHR records for clinical triggers for retrieving at least one database having patient affordability messaging, disease awareness messaging and clinical alerts updated information 370 and targeted ads for providing the physician with up-to-date information. A publishing device for displaying webpage winning bid ads within each EHR webpage 372 and patient condition messaging including product information from at least one database having advertiser product prescription data 374 of one embodiment.

### An Electronic Medical Record Advertising Platform

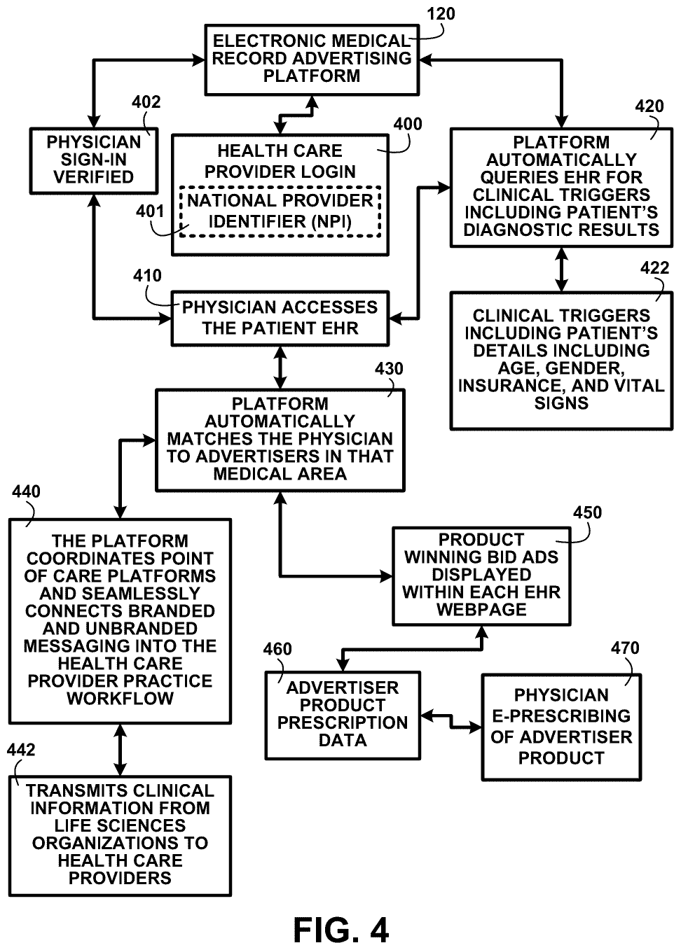

FIG. 4 shows a block diagram of an overview of an electronic medical record advertising platform of one embodiment. FIG. 4 shows an electronic medical record advertising platform 120 that integrates an EHR point of care platform with live science organization advertising. A health care provider login 400 includes the physician's national provider identifier (NPI) 401 to allow a physician sign-in to be verified 402 to gain access into the EHR point of care platform. When the physician accesses the patient EHR 410 the platform automatically queries EHR for clinical triggers including patient's diagnostic results 420.

Clinical triggers including patient's details including age, gender, insurance, and vital signs 422. The platform automatically matches the physician to advertisers in that medical area 430 and any specialty. The platform coordinates point of care platforms and seamlessly connects branded and unbranded messaging into the health care provider practice workflow 440. The platform transmits clinical information from life sciences organizations to health care providers 442. The product winning bid ads displayed within each EHR webpage 450 include an advertiser product prescription data 460. The advertiser product prescription data 460 aids the physician e-prescribing of advertiser product 470 of one embodiment.

### A Patient Calendar

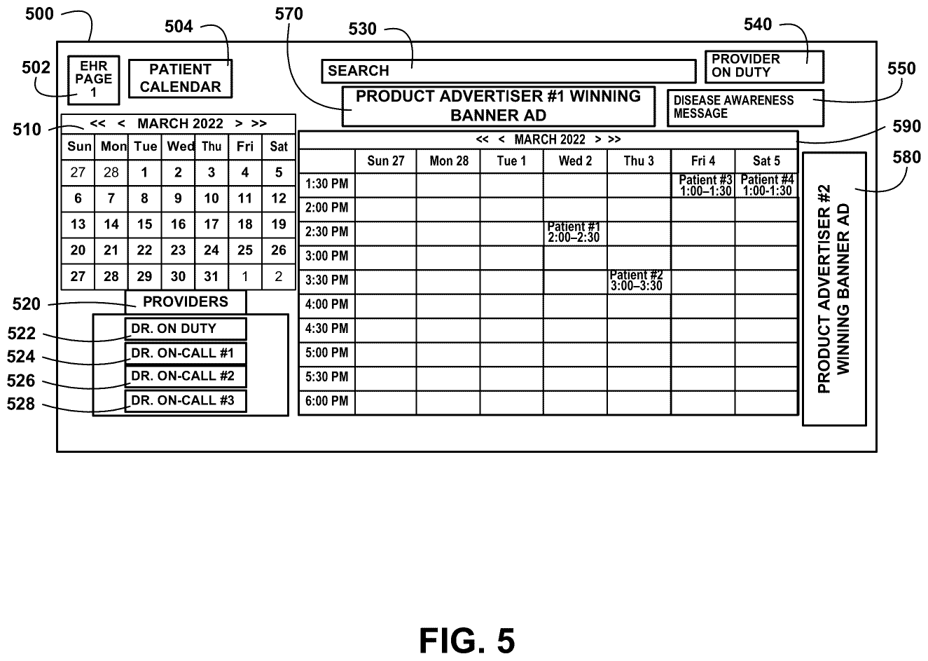

FIG. 5 shows for illustrative purposes only an example of a patient calendar of one embodiment. FIG. 5 shows in one embodiment, for example a physician patient workflow webpage 500. The example of a physician patient workflow webpage 500 is an EHR page 1 502 displaying a patient calendar 504. The patient calendar 504 shows the current month calendar 510 and a list of the point of care providers 520 including a Physician on duty 522, a Physician on call #1 524, a Physician on call #2 526 and a Physician on call #3 528. A search 530 tab allows the provider Physician on duty 540 to search the EHR of a patient.

In one embodiment, for example a space on the workflow webpage displays a disease awareness message 550 the physician can activate to get updated information on a patient's condition. The workflow webpage is also displaying a product advertiser #1 winning banner ad 570 and a product advertiser #2 winning banner ad 580 on the provider patient appointment calendar 590. The display of the ads saves the physician time by making the information on the product readily available of one embodiment.

### A Patient Chart

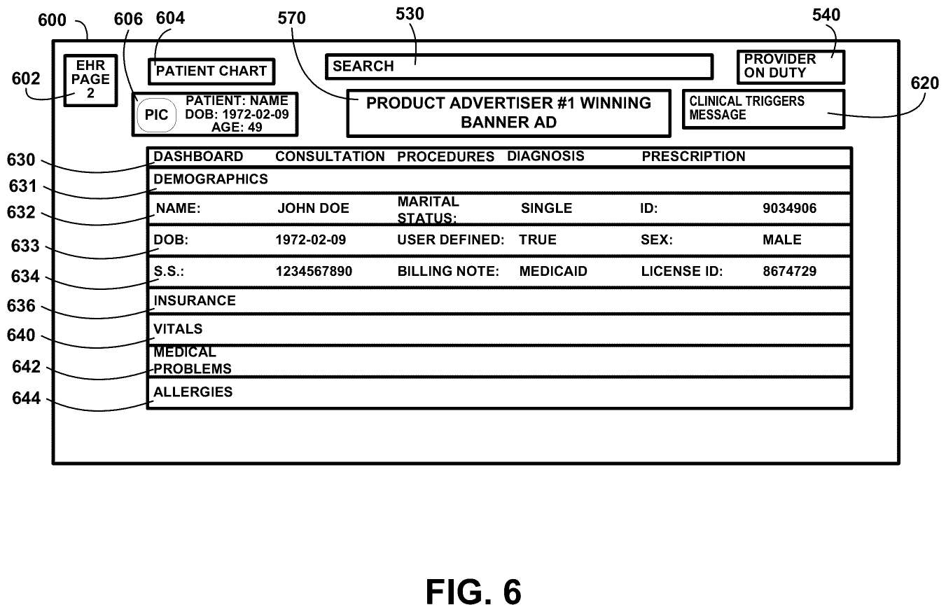

FIG. 6 shows for illustrative purposes only an example of a patient chart of one embodiment. FIG. 6 shows in one embodiment, for example a physician patient continuing workflow webpage 600 for example EHR page 2 602. In one embodiment, for example the physician continues the patient check-up and makes entries on the patient chart 604. Information associated with clinical triggers for this specific patient is seen on the patient chart 604 including the patient name date of birth 1972-02-09 age 49 and picture of the patient 606. The search 530 tab is used by the provider Physician on duty 540 to call up messages.

In one embodiment, for example a clinical triggers message 620 lists a number of clinical triggers used to select a product advertiser #1 winning banner ad 570. A workflow webpage includes a dashboard, consultation, procedures, diagnosis; prescription 630 displays the physician can activate any of the categories. The patient chart 604 is displaying the patient demographics 631 including name John Doe, marital status single, id 9034906 632, DOB 1972-02-09, user defined true, sex male 633 and S.S. 1234567890, billing note Medicaid, license id 8674729 634. Also available to the physician and critical targeting queries are the patient insurance 636, vitals 640, medical problems 642, and allergies 644 of one embodiment.

Providing a Physician with Information

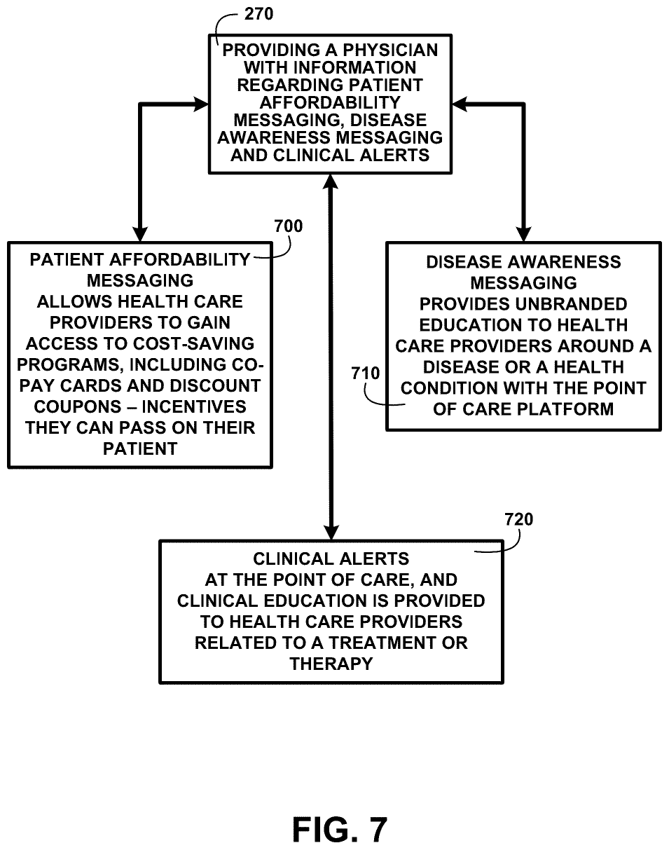

FIG. 7 shows a block diagram of an overview of providing a physician with information of one embodiment. FIG. 7 shows providing a physician with information regarding patient affordability messaging; disease awareness messaging and clinical alerts 270. Patient affordability messaging allows health care providers to gain access to cost-saving programs, including co-pay cards and discount coupons—incentives they can pass on their patient 700. Disease awareness messaging provides unbranded education to health care providers around a disease or a health condition with the point of care platform 710. Clinical alerts at the point of care and clinical education are provided to health care providers related to a treatment or therapy 720 of one embodiment.

### Creation Ad Copy in Various Formats

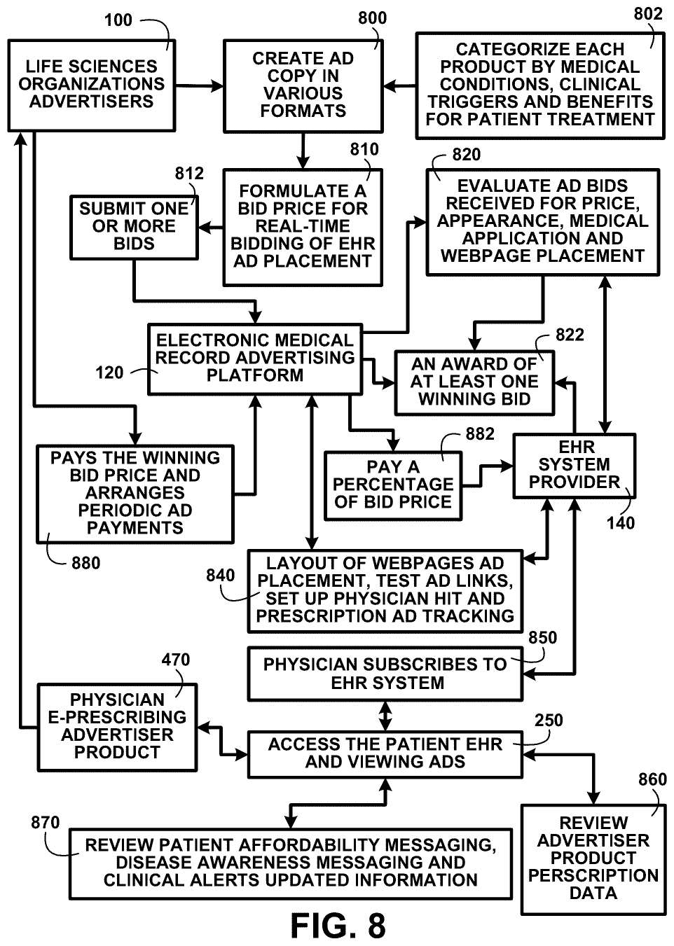

FIG. 8 shows a block diagram of an overview of creation of ad copy in various formats of one embodiment. FIG. 8 shows the life sciences organizations advertisers 100 who create ad copy in various formats 800. The life sciences organizations advertisers 100 categorize each product by medical conditions, clinical triggers, and benefits for patient treatment 802. The advertisers formulate a bid price for real-time bidding of EHR ad placement 810. The life sciences organizations advertisers 100 submit one or more bids 812 to the electronic medical record advertising platform 120.

The electronic medical record advertising platform 120 and EHR system provider 140 evaluate ad bids received for price, appearance, medical application, and webpage placement 820. After the evaluations, an award of at least one winning bid 822 is made. The EHR system provider 140 process the layout of webpages ad placement, test ad links, set up physician hit and prescription ad tracking 840. A physician subscribes to EHR system 850 to access the patient EHR and viewing ads 250 and review advertiser product prescription data 860.

The physician may also review patient affordability messaging, disease awareness messaging and clinical alerts updated information 870. The information assists the physician e-prescribing advertiser product 470. The life sciences organizations advertisers 100 pays the winning bid price and arranges periodic ad payments 880 to the electronic medical record advertising platform 120 and the platform automatically process the payment to pay a percentage of bid price 882 to the EHR system provider 140 of one embodiment.

### Providing Latest Medical Information to Health Care Providers

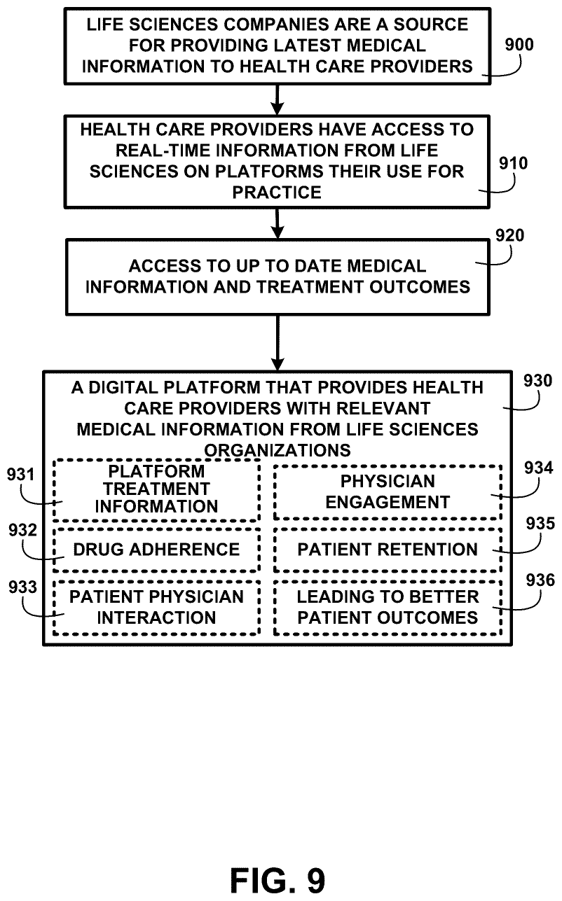

FIG. 9 shows a block diagram of an overview of providing latest medical information to health care providers of one embodiment. FIG. 9 shows the life sciences companies are a source for providing latest medical information to health care providers 900. The health care providers have access to real-time information from life sciences on platforms they use for their practice 910. The physician does not have to spend time researching data since the physician has through the EHR system, access to up to date medical information and treatment outcomes 920.

The integrated physician provides a digital platform that provides health care providers with relevant medical information from life sciences organizations 930. The life sciences information and ad integration are platform treatment information 931, improve drug adherence 932, add to patient physician interaction 933, and provide physician engagement 934, patient retention 935 all leading to better patient outcomes 936 of one embodiment.

### Patient Affordability Messaging

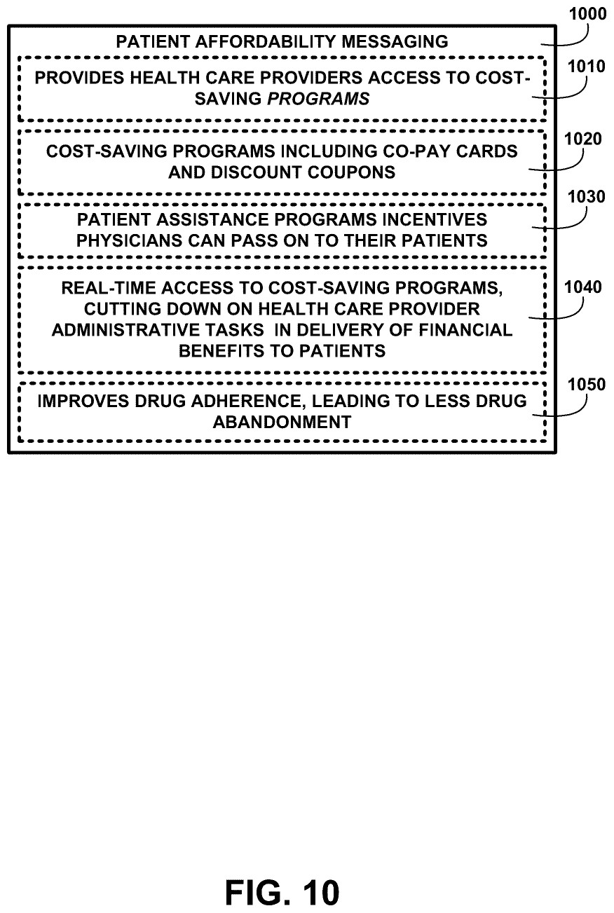

FIG. 10 shows a block diagram of an overview of patient affordability messaging of one embodiment. FIG. 10 shows patient affordability messaging 1000 that provides health care providers access to cost-saving programs 1010. The cost-saving programs including co-pay cards and discount coupons 1020 are patient assistance programs incentives they can pass on to their patients 1030. Real-time access to cost-saving programs, cutting down on health care provider administrative tasks in delivery of financial benefits to patients 1040 improves drug adherence, leading to less drug abandonment 1050 of one embodiment.

### Clinical Alerts

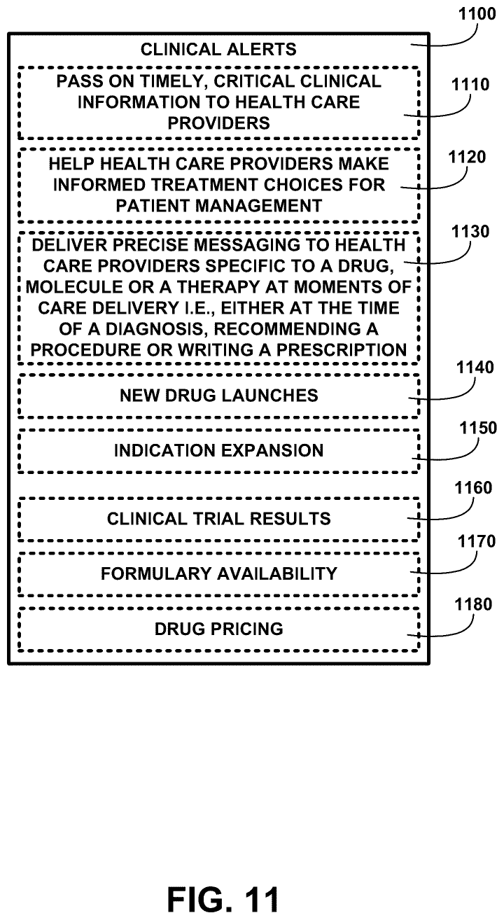

FIG. 11 shows a block diagram of an overview of clinical alerts of one embodiment. FIG. 11 shows clinical alerts 1100 used to pass on timely, critical clinical information to health care providers 1110. The clinical alerts 1100 help health care providers make informed treatment choices for patient management 1120 without having to research up to date data. The clinical alerts 1100 deliver precise messaging to health care providers specific to a drug, molecule, or a therapy at moments of care delivery i.e., either at the time of a diagnosis, recommending a procedure or writing a prescription 1130. Physicians are kept aware of new drug launches 1140, indication expansion 1150, clinical trial results 1160, formulary availability 1170 and drug pricing 1180 of one embodiment.

### Disease Awareness Messaging

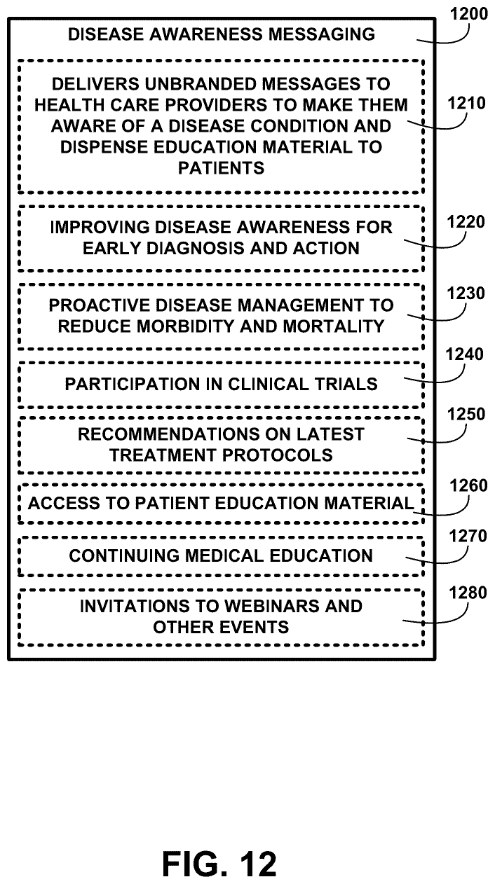

FIG. 12 shows a block diagram of an overview of disease awareness messaging of one embodiment. FIG. 12 shows disease awareness messaging 1200 that delivers unbranded messages to health care providers to make them aware of a disease condition and dispense education material to patients 1210. This messaging is improving disease awareness for early diagnosis and action 1220. This messaging is proactive disease management to reduce morbidity and mortality 1230. A physician may determine this patient may be a candidate for participation in clinical trials 1240. It informs the physician of recommendations on latest treatment protocols 1250. The patient is more engaged with the physician with access to patient education material 1260. The physician is better prepared with readily available continuing medical education 1270 and invitations to webinars and other events 1280 of one embodiment.

Ads and Information Integrated within the Practice Workflow

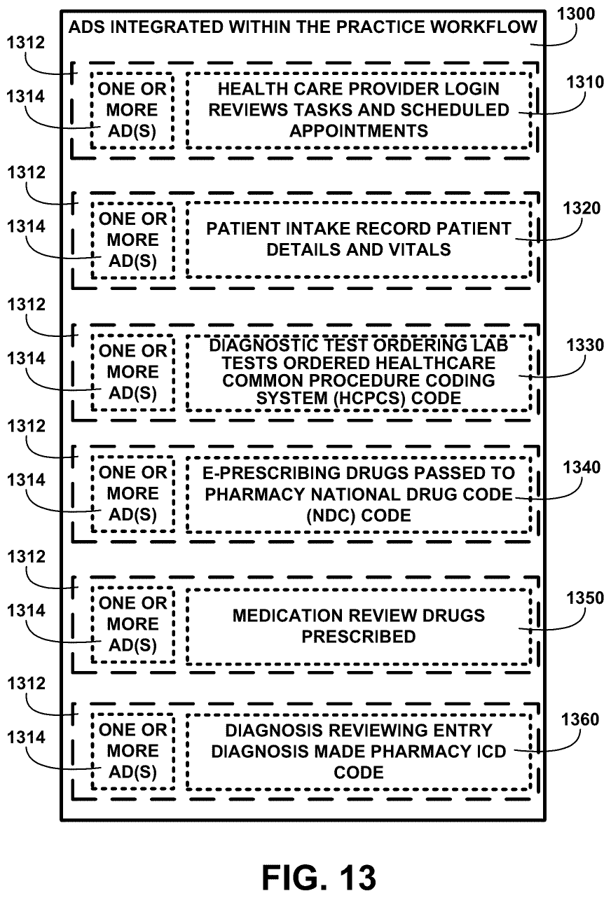

FIG. 13 shows a block diagram of an overview of information integrated within the practice workflow of one embodiment. FIG. 13 shows ads integrated within the practice workflow 1300. An initial webpage may include when a health care provider login reviews tasks and scheduled appointments 1310 on the practice workflow webpage 1312 with one or more ad(s) 1314 placed in the webpage. Patient intake records patient details and vitals 1320 on the practice workflow webpage 1312 with one or more ad(s) 1314 placed in the webpage.

Diagnostic test ordering lab tests ordered Healthcare Common Procedure Coding System (HCPCS) code 1330 on the practice workflow webpage 1312 with one or more ad(s) 1314 placed in the webpage. E-prescribing drugs passed to pharmacy National Drug Code (NDC) code 1340 on the practice workflow webpage 1312 with one or more ads) 1314 placed in the webpage. Medication review drugs prescribed 1350 on the practice workflow webpage 1312 with one or more ad(s) 1314 placed in the webpage. Diagnosis reviewing entry diagnosis made pharmacy International Classification of Diseases (ICD) code 1360 on the practice workflow webpage 1312 with one or more ad(s) 1314 placed in the webpage of one embodiment.

### Selecting Precise Message Based on the Workflow

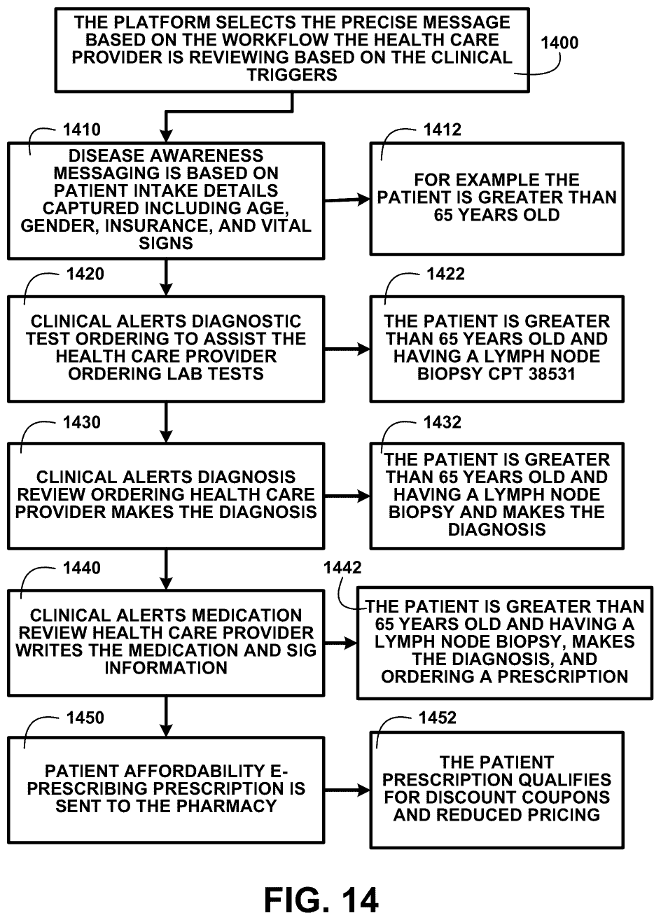

FIG. 14 shows a block diagram of an overview of selecting precise message based on the workflow of one embodiment. FIG. 14 shows the platform selects the precise message based on the workflow the health care provider is reviewing based on the clinical triggers 1400. In one embodiment, for example disease awareness messaging is based on patient intake details captured including age, gender, insurance, and vital signs 1410 for example, the patient is greater than 65 years old 1412.

One workflow webpage includes clinical alerts diagnostic test ordering to assist the health care provider ordering lab tests 1420. In one embodiment, for example this workflow shows the patient is greater than 65 years old and having a lymph node biopsy 1422. Clinical alerts diagnosis review ordering assists the health care provider in making the diagnosis 1430. The patient is greater than 65 years old and having a lymph node biopsy and makes the diagnosis 1432. Clinical alerts medication review assists the health care provider with the medication information 1440. The patient is greater than 65 years old and having a lymph node biopsy, makes the diagnosis, and ordering a prescription 1442. In one embodiment, for example the patient affordability e-prescribing prescription is sent to the pharmacy 1450. The patient prescription qualifies for discount coupons and reduced pricing 1452 of one embodiment.

### Outcomes

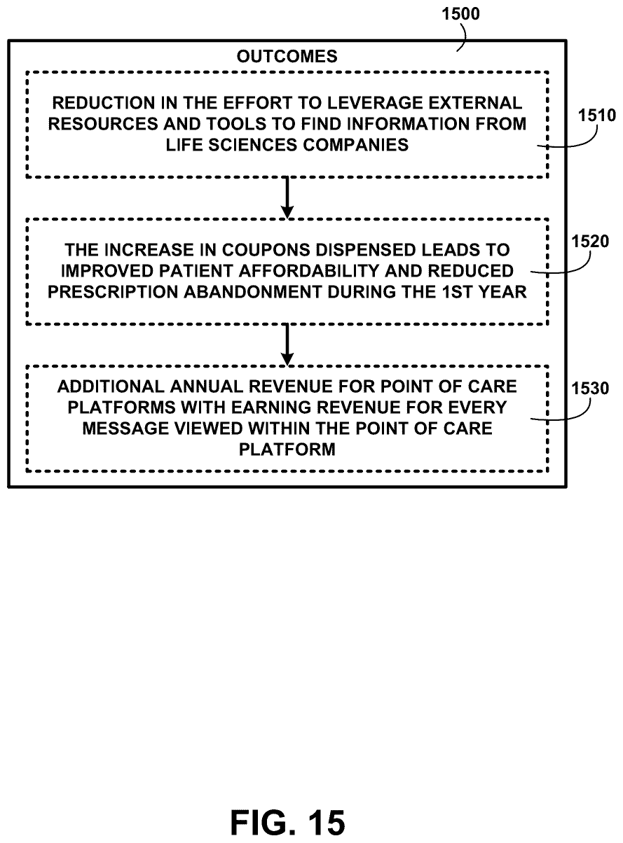

FIG. 15 shows a block diagram of an overview of outcomes of one embodiment. FIG. 15 shows outcomes 1500 benefiting health care providers. In one embodiment, for example a benefit is a reduction in the effort to leverage external resources and tools to find information from life sciences companies 1510. In another embodiment, for example the increase in coupons dispensed leads to improved patient affordability and reduced prescription abandonment during the 1st year 1520. In other embodiments, for example benefits include additional annual revenue for point of care platforms with earning revenue for every message viewed within the point of care platform 1530 of one embodiment.

### HIPAA Compliance

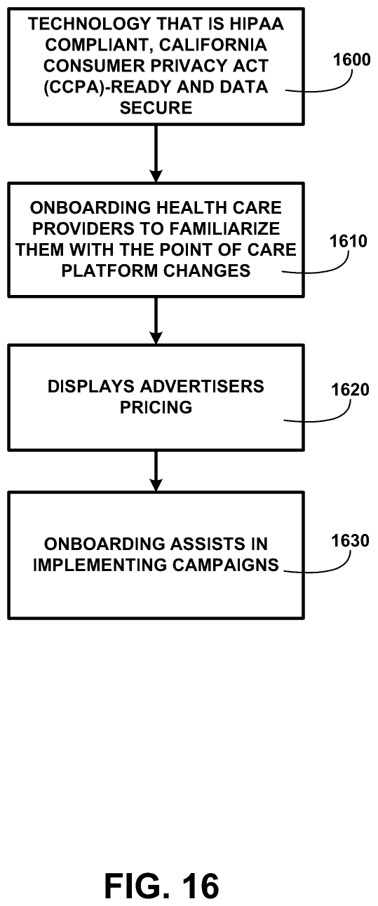

FIG. 16 shows a block diagram of an overview of HIPAA compliance of one embodiment. FIG. 16 shows technology that is HIPAA compliant, California Consumer Privacy Act (CCPA)-READY and data secure 1600. Onboarding health care providers to familiarize them with the point of care platform changes 1610. The electronic medical record advertising platform 120 of FIG. 1 displays advertisers pricing 1620. Onboarding assists in implementing campaigns 1630 of one embodiment.

Engaging with Health Care Providers

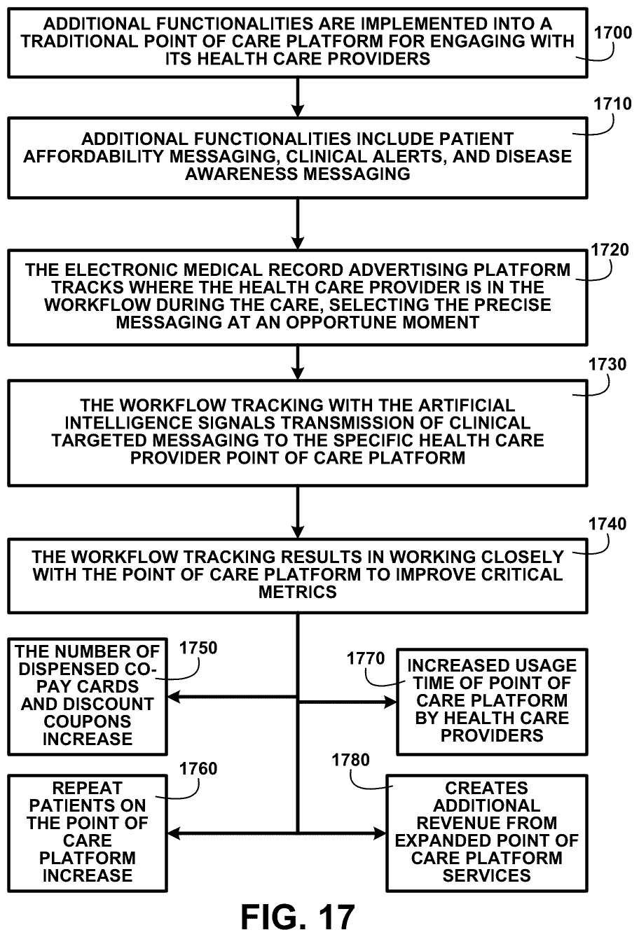

FIG. 17 shows a block diagram of an overview of engaging with health care providers of one embodiment. FIG. 17 shows additional functionalities are implemented into a traditional point of care platform for engaging with its health care providers 1700.

Additional functionalities include patient affordability messaging, clinical alerts, and disease awareness messaging 1710. The electronic medical record advertising platform tracks where the health care provider is in the workflow during the care, selecting the precise messaging at an opportune moment 1720. The workflow tracking with the artificial intelligence signals transmission of clinical targeted messaging to the specific health care provider point of care platform 1730. The workflow tracking results is working closely with the point of care platform to improve critical metrics 1740. The number of dispensed co-pay cards and discount coupons increase 1750; repeat patients on the point of care platform increase 1760; and increased usage time of point of care platform by health care providers 1770 creates additional revenue from expanded point of care platform services 1780 of one embodiment.

### Onboarding Process

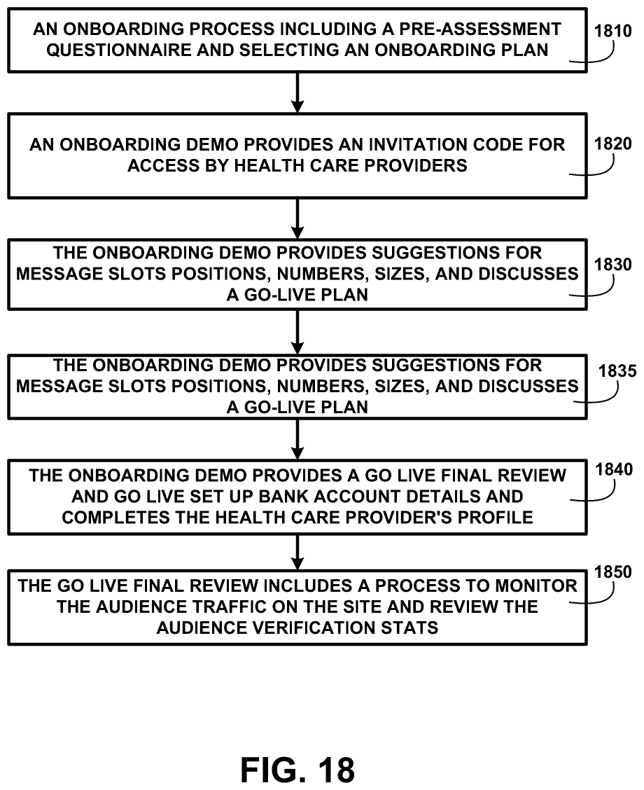

FIG. 18 shows a block diagram of an overview of an onboarding process of one embodiment. FIG. 18 shows an onboarding process including a pre-assessment questionnaire and selecting an onboarding plan 1810. An onboarding demo provides an invitation code for access by health care providers 1820. The onboarding demo system test with a review of the platform onboarding 1830. The onboarding demo provides suggestions for message slots positions, numbers, sizes, and discusses a go-live plan 1835. The onboarding demo provides a go live final review and goes live set up bank account details and completes the health care provider's profile 1840. The go live final review includes a process to monitor the audience traffic on the site and review the audience verification stats 1850 of one embodiment.

### Partnering

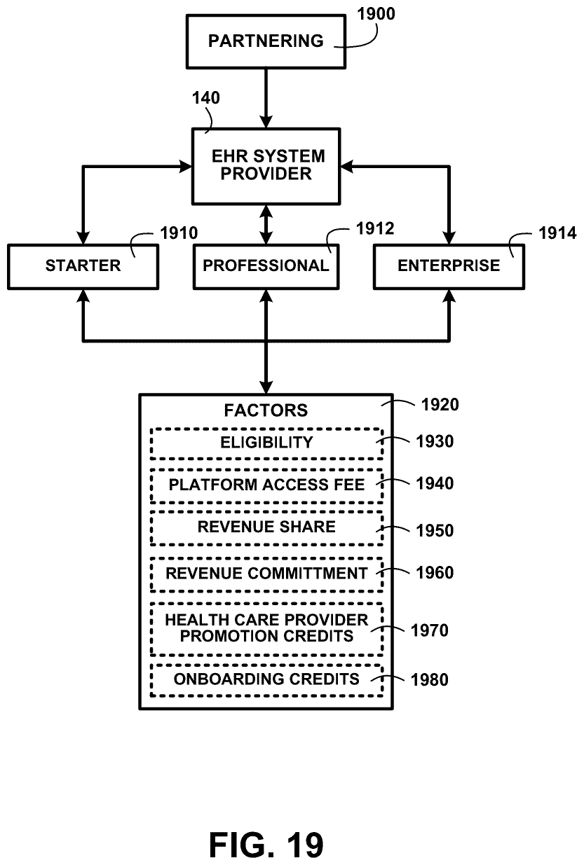

FIG. 19 shows a block diagram of an overview of partnering of one embodiment. FIG. 19 shows partnering 1900 between the electronic medical record advertising platform 120 of FIG. 1 and the EHR system provider 140. The EHR system provider 140 partnerships may be classified by the numbers of subscribers as a starter 1910, professional 1912, and enterprise 1914. Partnering 1900 factors 1920 are differentiated for the classification and partnering structure include eligibility 1930, platform access fee 1940, revenue share 1950, revenue commitment 1960, health care provider promotion credits 1970, and onboarding credits 1980 of one embodiment.

### Targeted EHR Webpages to Bid on

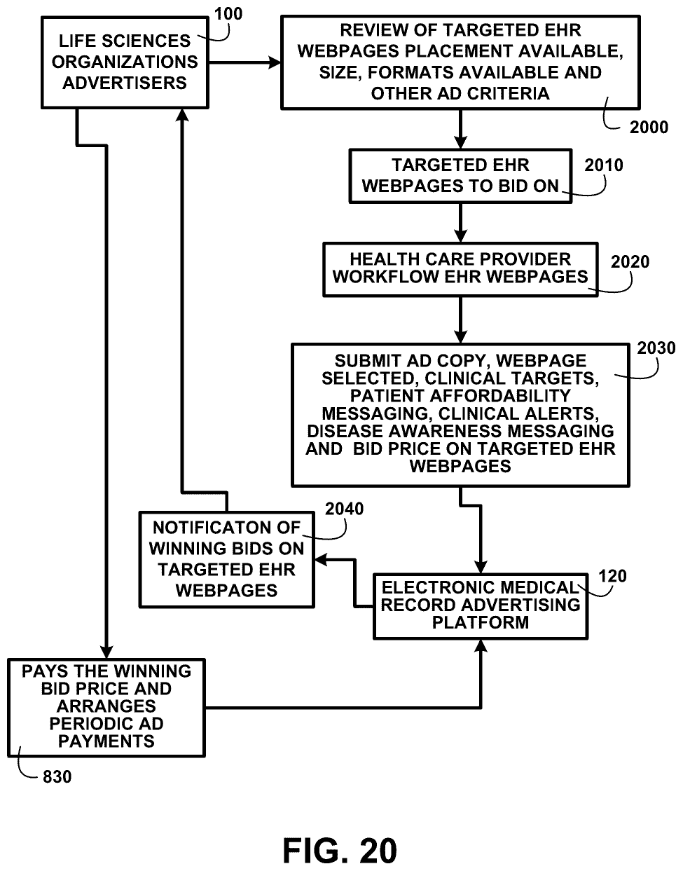

FIG. 20 shows a block diagram of an overview of targeted EHR webpages to bid on of one embodiment. FIG. 20 shows the life sciences organizations advertisers 100 review of targeted EHR webpages placement available, size, formats available and other ad criteria 2000 to decide which targeted EHR webpages to bid on 2010. Once the life sciences organizations advertisers 100 selects one or more health care provider workflow EHR webpages 2020 they will submit ad copy, webpages selected, clinical targets, patient affordability messaging, clinical alerts, disease awareness messaging and bid price on targeted EHR webpages 2030. The electronic medical record advertising platform 120 communicates a notification of winning bids on targeted EHR webpages 2040 to the life sciences organizations advertisers 100. The winning life sciences organizations advertiser 100 pays the winning bid price and arranges periodic ad payments 830 of one embodiment.

### Establishing Health Care Provider Workflow Web Pages

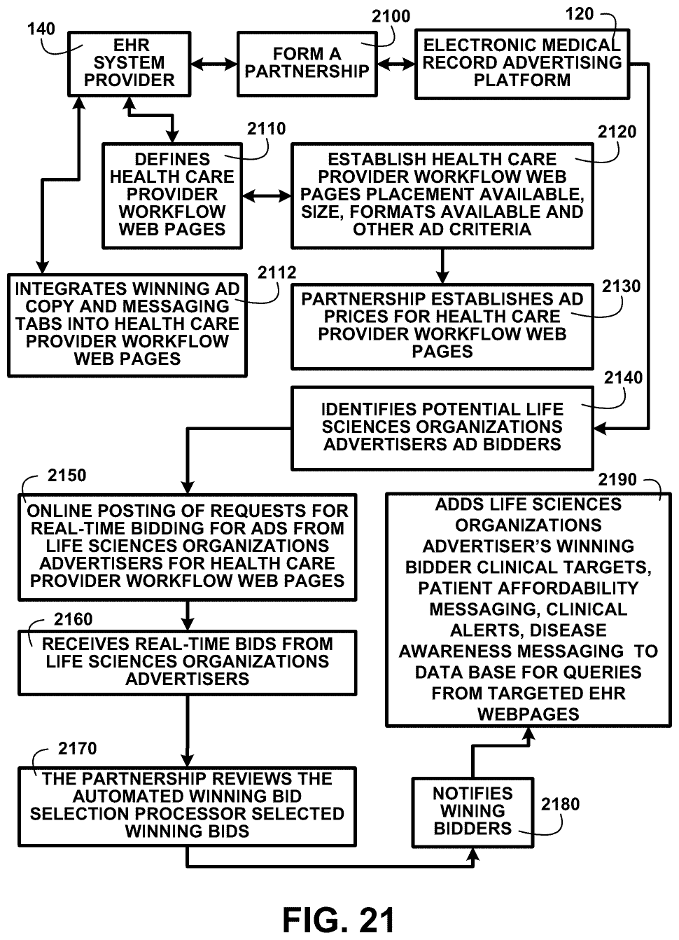

FIG. 21 shows a block diagram of an overview of establishing health care provider workflow web pages of one embodiment. FIG. 21 shows the EHR system provider 140 agrees to form a partnership 2100 with the electronic medical record advertising platform 120. The EHR system provider 140 defines health care provider workflow web pages 2110 and integrates winning ad copy and messaging tabs into health care provider workflow web pages 2112.

The EHR system provider 140 will establish health care provider workflow web pages placement available, size, formats available and other ad criteria 2120. The partnership establishes ad prices for health care provider workflow web pages 2130. The electronic medical record advertising platform 120 identifies potential life sciences organizations advertiser ad bidders 2140 with products targeting patient conditions. The electronic medical record advertising platform 120 proceeds with online posting of requests for real-time bidding for ads from life sciences organizations advertisers for health care provider workflow web pages 2150.

The electronic medical record advertising platform 120 receives real-time bids from life sciences organizations advertisers 2160. The partnership reviews the automated winning bid selection processor selected winning bids 2170. The electronic medical record advertising platform 120 notifies the winning bidders 2180. The partnership adds life sciences organizations advertisers winning bidder clinical targets, patient affordability messaging, clinical alerts, and disease awareness messaging to data base for queries from targeted EHR webpages 2190 of one embodiment.

Physician e-Prescribing Medication from Product Advertiser

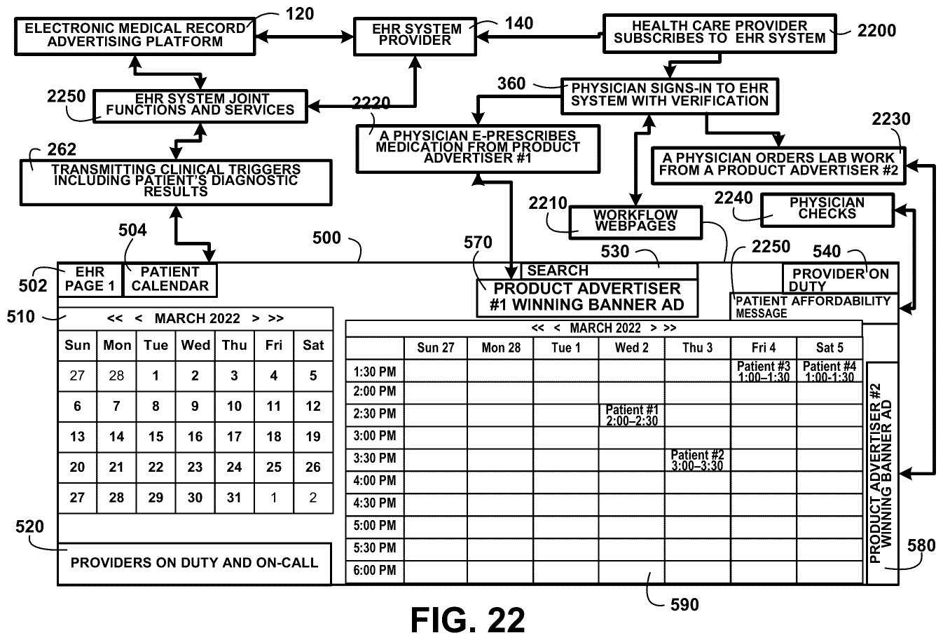

FIG. 22 shows a block diagram of an overview of physician e-prescribing medication from product advertiser of one embodiment. FIG. 22 shows the electronic medical record advertising platform 120 and EHR system provider 140 integrated platforms provides expanded capability for a point of care platform. A health care provider subscribes to EHR system 2200. The physician signs-in to EHR system with verification 360 and begins accessing workflow webpages 2210. In one embodiment, for example a physician e-prescribes medication from product advertiser #1 2220.

A physician orders lab work from a product advertiser #2 2230. The physician checks 2240 a patient affordability message 2250 to receive updated information on the patient's cost-saving programs. In one embodiment, for example the physician patient workflow webpage 500 EHR page 1 502 displays the patient calendar 504, current month calendar 510, providers 520, and search 530 tab. The provider Physician on duty 540 can see and tap the product advertiser #1 winning banner ad 570 and product advertiser #2 winning banner ad 580 to learn more about those products. The provider patient appointment calendar 590 data is transmitting clinical triggers including patient's diagnostic results 262 in one aspect of the EHR system joint functions and services 2250 of one embodiment.

The foregoing has described the principles, embodiments and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A method, comprising
providing real-time life science advertiser bids on programmatic media targeted to an electronic health record (EHR) system via an electronic medical record advertising platform;
providing life science advertiser product ad bids that target clinical triggers;
providing access of a patient EHR by a user;
transmitting electronically patient privacy regulatory compliant clinical triggers from the patient electronic health record;
providing a user with information regarding a patient's clinical trigger electronically selected including patient affordability messaging, disease awareness messaging and clinical alerts;
providing a display of life science advertiser product winning bid ads within each EHR webpage;
providing life science advertiser product prescription data; and
providing networking connectivity capabilities between life science advertisers, EHR system delivery operators and users to facilitate patient care.

2. The method of claim 1, further comprising providing verification of a user's electronic log-in to an electronic platform using the physician's national provider identifier.

3. The method of claim 1, further comprising providing to an electronic platform verified logged-in user a patient's diagnostic results with patient privacy regulatory compliant clinical triggers.

4. The method of claim 1, further comprising integrating electronically superimposing into a user's patient EHR workflow webpages a display of at least one life science advertiser product winning bid ad, patient affordability messaging, disease awareness messaging and clinical alerts, and life science advertiser product prescription data based on at least one patient clinical trigger.

5. The method of claim 1, wherein selecting electronically patient privacy regulatory compliant clinical triggers from the patient EHR including patient demographics.

6. The method of claim 1, wherein providing patient privacy regulatory compliant clinical triggers from the patient EHR includes patient insurance, vitals, medical problems and allergies.

7. The method of claim 1, wherein providing life science advertiser product prescription data aids the user when e-prescribing of an advertiser medication product.

8. An apparatus, comprising
an electronic medical record advertising platform configured for receiving life science advertiser real-time bids for electronic health record (EHR) system workflow webpage ad placement;
at least one database coupled to the electronic medical record advertising platform configured for recording life science advertiser ads, user-level data, messaging, product prescription data, clinical alerts and patient privacy regulatory compliant clinical triggers from a patient electronic health record;
at least one data query processor coupled to the electronic medical record advertising platform configured for accessing at least the patient privacy regulatory compliant clinical triggers from the at least one database;
at least one electronic publishing device coupled to the electronic medical record advertising platform configured for displaying workflow webpage winning bid ads and messaging within each EHR workflow webpage;
at least one digital tracking device coupled to the electronic medical record advertising platform configured for tracking and recording EHR system workflow webpages ad hits, messaging traffic and prescriptions of life science advertisers products; and
an electronic medical record advertising app wirelessly coupled to the electronic medical record advertising platform computer configured for accessing EHR system integrated targeted ads for electronic medical record advertising.

9. The apparatus of claim 8, further comprising the data mining processor coupled to the electronic medical record advertising platform computer configured for extracting patient privacy regulatory compliant clinical triggers from a patient EHR including patient demographics.

10. The apparatus of claim 8, further comprising the data mining processor coupled to the electronic medical record advertising platform computer configured for extracting patient privacy regulatory compliant clinical triggers from a patient EHR including insurance, vitals, medical problems and allergies.

11. The apparatus of claim 8, further comprising the at least one electronic publishing device coupled to the electronic medical record advertising platform configured for providing an accessible life science advertiser product prescription data tab to access the prescription data to aid the user e-prescribing of an advertiser medication product.

12. The apparatus of claim 8, further comprising a user sign-in system coupled to the EHR system configured for verifying a user sign-in with a physician's national provider identifier.

13. The apparatus of claim 8, wherein the patient privacy regulatory compliant clinical triggers includes patient diagnostic results.

14. The apparatus of claim 8, wherein the at least one electronic publishing device coupled to the electronic medical record advertising platform configured for displaying workflow webpage messaging includes patient affordability messaging, disease awareness messaging and clinical alerts, and life science advertiser product prescription data based on clinical triggers.

15. An apparatus, comprising
a network platform configured for receiving advertiser real-time bids for electronic health record (EHR) system workflow webpage ad placement;
at least one webpage graphic processor coupled to the network platform configured for integrating ad placement into EHR system workflow webpage formats;
a data mining processor for extracting patient privacy regulatory compliant clinical triggers from a patient EHR;
at least one electronic publishing device coupled to the network platform configured for displaying workflow webpage ads and messaging within each workflow webpage; and
an electronic medical record advertising application wirelessly coupled to the network platform computer configured for accessing an EHR system having integrated targeted ads for electronic medical record advertising.

16. The apparatus of claim 15, further comprising at least one electronic brand dashboard coupled to the network platform configured for transmitting EHR system workflow webpages ad hits, messaging traffic and prescriptions of life science advertisers' products to a life science advertiser.

17. The apparatus of claim 15, further comprising at least one database coupled to the network platform configured for recording advertiser ads, messaging, product prescription data, clinical alerts and patient privacy regulatory compliant clinical triggers from a patient EHR.

18. The apparatus of claim 15, further comprising at least one digital tracking device coupled to the network platform configured for tracking and recording EHR system workflow webpages ad hits, messaging traffic and user e-prescriptions of life science advertisers' products.

19. The apparatus of claim 15, further comprising a user sign-in system coupled to the network platform computer configured for verifying a user sign-in with a physician's national provider identifier.

20. The apparatus of claim 15, further comprising the at least one electronic publishing device coupled to the network platform is configured for integrating into the display of patient EHR workflow webpages patient affordability messaging, disease awareness messaging and clinical alerts, and advertiser product prescription data based on clinical triggers.
